(19) United States
(12) Patent Application Publication
(10) Pub. No.: US 2020/0101238 A1
(43) Pub. Date: Apr. 2, 2020

(54) SYRINGE WITH A LUER LOCK CONNECTOR

(71) Applicant: Schott Schweiz AG, St. Gallen (CH)

(72) Inventors: Judith Auerbach, Niederteufen (CH); Bastian Fischer, St. Gallen (CH); Helena Derksen, St. Gallen (CH); Jens Meiss, St. Gallen (CH)

(73) Assignee: Schott Schweiz AG, St. Gallen (CH)

(21) Appl. No.: 16/712,042

(22) Filed: Dec. 12, 2019

Related U.S. Application Data
(63) Continuation of application No. PCT/EP2018/063643, filed on May 24, 2018.

(30) Foreign Application Priority Data
Jun. 12, 2017 (DE) 10 2017 112 823.9

(51) Int. Cl. A61M 5/34; A61K 31/05; A61M 5/31

(52) U.S. Cl. CPC A61M 5/347; A61M 2005/3143; A61M 5/3137; A61K 31/05

(57) ABSTRACT

A syringe includes a syringe body, which has a cylindrical portion and, adjoining the latter, a frustoconical portion, and a Luer lock connector, which has a Luer collar with an inner thread and a Luer cone with a through-channel. A bottom surface is formed between the Luer collar and Luer cone. The syringe body and the Luer lock connector are formed in one piece and are made of a cyclic olefin resin. The Luer collar has reinforcing ribs on its outer face.

### CROSS REFERENCE TO RELATED APPLICATIONS

This is a continuation of PCT application No. PCT/EP2018/063643, entitled “SYRINGE WITH A LUER LOCK CONNECTOR”, filed May 24, 2018, which is incorporated herein by reference.

### BACKGROUND OF THE INVENTION

### 1. Field of the Invention

The present invention relates to a syringe with a Luer lock connector, and to a pharmaceutical product comprising the syringe. The syringe body and the Luer lock connector of the syringe are formed in one piece and are made of a cyclic olefin resin.

### 2. Description of the Related Art

Syringes with a Luer lock connector are known from the prior art. Items of medical equipment such as needles, tubes or other attachment parts can be joined to the syringe via the Luer lock connector. The elements of a Luer lock connector are standardized in ISO 594/1, 2 and ISO 80369-7 in order to ensure an optimal fit with good leak-tightness and strength. A Luer lock connector comprises, on the syringe side, a male Luer cone with a central through-channel for the medicament, which Luer cone is surrounded by a Luer collar with an inner thread. The attachment, which is joined on, has a female Luer fitting, of which the outer face engages in the thread of the Luer collar when screwed in and is thus tightly connected to the Luer cone.

Many different attachments are in practice joined to a syringe with a Luer lock connector, since many manufacturers of the attachments use their own, non-standardized connector parts. Thus, the outer face of an attachment can, for example, have a continuous circumferential thread corresponding to the Luer collar, or it may instead be provided only over a short axial portion with elements which engage in the thread of the Luer collar.

It has been found that, even when the requirements of ISO 594-1, 2 and ISO 80369-7 are met, various problems can affect a Luer lock connection, since cyclic olefin polymers have a brittle behavior and, if the force applied by the attachment is too great and not standard, fractures may occur. This applies in particular if an attachment is screwed in with too great a torque. Very high local forces can arise inside the Luer lock connector, particularly if the attachment does not have a complete circumferential outer thread. Stress fractures can then arise both in the region of the inner Luer cone and in the region of the outer Luer collar, and these stress fractures can lead to shearing-off of the Luer collar, or they can even run into the syringe body such that the leak-tightness of the syringe body or of the Luer connection is compromised.

With highly viscous medicaments in particular, a high internal pressure also places a strain on syringe body and Luer lock connector during emptying of the syringe. The stability of the connection can be additionally weakened by additional treatments, e.g. autoclaving.

U.S. Pat. No. 8,038,132 likewise addresses the technical problem of the lack of strength of a Luer lock connector. It proposes that the outer face of the Luer cone be provided with a mechanical abutment, which limits the depth to which an attachment can be screwed in. Moreover, a cavity under the abutment is proposed into which the outer part of the attachment, which engages in the Luer thread, can extend. A disadvantage is that the Luer collar is still at risk of breaking, and a Luer lock connector of the geometry shown can be produced only with difficulty.

EP 0838229 proposes providing the inner thread of the Luer collar with an abutment which prevents an attachment from being screwed in too far, for example an attachment according to FIG. 3 of EP 0838229. It is alternatively proposed that the wall thickness between Luer lock connector and syringe body be significantly increased. It is further proposed that an annular elevation or step be provided at the foot of the Luer collar, which elevation or step is intended to prevent lateral outward deflection of the female attachment according to FIG. 4B. While the first measure cannot reliably prevent an elastomeric connector part from being screwed in too far and cannot easily be realized by injection molding, the second proposed solution can lead to problems or increased costs in the manufacture by injection molding. The third proposed solution is closely bound to the particular type of female connector according to FIG. 4B and, in conjunction with elastomeric or differently configured attachments, loading of the Luer collar cannot be reliably excluded.

What is needed in the art is a syringe with a Luer lock connector formed in one piece therewith, which syringe, in combination with various attachments used in practice, ensures a permanent leak-tight and stable Luer connection and can be easily produced by injection molding.

### SUMMARY OF THE INVENTION

In some exemplary embodiments provided according to the present invention, a syringe includes a syringe body, which has a cylindrical portion and, adjoining the latter, a frustoconical portion, and a Luer lock connector, which has a Luer collar with an inner thread and a Luer cone with a through-channel. A bottom surface is formed between the Luer collar and Luer cone. The syringe body and the Luer lock connector are formed in one piece and are made of a cyclic olefin resin. The Luer collar has reinforcing ribs on its outer face.

### BRIEF DESCRIPTION OF THE DRAWINGS

The above-mentioned and other features and advantages of this invention, and the manner of attaining them, will become more apparent and the invention will be better understood by reference to the following description of embodiments of the invention taken in conjunction with the accompanying drawings, wherein:

FIG. 1 illustrates a perspective sectional view of a prior art Luer lock connector with screwed-in attachment;

FIG. 2 illustrates prior art attachments as per ISO 594-2;

FIG. 3 illustrates a longitudinal section through an exemplary embodiment of a syringe provided according to the present invention;

FIG. 4 illustrates a perspective sectional view of a Luer lock connector of an exemplary embodiment of a syringe provided according to the present invention with screwed-in attachment;

FIG. 5 illustrates a horizontal cross section through a Luer lock connector of an exemplary embodiment of a syringe provided according to the present invention; and

FIG. 6 illustrates a horizontal cross section through a reinforcing rib.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION OF THE INVENTION

In some exemplary embodiments provided according to the present invention, a syringe includes a syringe body, which has a cylindrical portion and a frustoconical portion adjoined to the cylindrical portion, and a Luer lock connector, which has a Luer collar with an inner thread and a Luer cone with a through-channel. A bottom surface is formed between the Luer collar and Luer cone. The syringe body and the Luer lock connector are formed in one piece and are made of a cyclic olefin resin. The Luer collar has reinforcing ribs on its outer face.

It has been surprisingly found that the reinforcing ribs on the Luer cone are not only able to significantly increase the stiffness of the Luer collar, but that the propagation of stress fractures from the syringe body can also be diverted by the reinforcing ribs. In cylindrical Luer collars of the prior art without reinforcing ribs, the propagation of fractures goes relatively unimpeded, such that the entire Luer cone may shear off or separate, whereas fractures in Luer collars with reinforcing ribs surprisingly come to a stop at the reinforcing ribs. Moreover, an increase in the wall thickness of the Luer collar has shown no effect comparable to the reinforcing ribs. Contrary to the assumption that the formation of fractures is slowed down by the ribs, it was also discovered that the number of fractures can also be reduced by the reinforcing ribs.

The syringe body serves to receive a pharmaceutical and is designed to interact with a stopper. It is made at least for the most part, and in some embodiments completely, of a cyclic olefin resin such as cyclic olefin copolymer (COC) or cyclic olefin copolymer (COP). Trade names of these cyclic olefin resins are, for example, Zeonor®, Zeonex® and ARTON®.

The cylindrical portion of the syringe body serves to receive the pharmaceutical and is designed to interact with a stopper. The inner surface of the cylindrical portion of the syringe body can have a sliding layer which ensures low static friction and sliding friction in connection with a stopper. The outer and/or inner faces can also be provided with barrier layers which, for example, reduce the diffusion of oxygen. The wall thickness of the syringe body is typically in a range of 0.7 mm to 2.5 mm.

The frustoconical portion of the syringe body represents the region of the syringe body which connects the cylindrical portion of the syringe body to the Luer lock connector and the through-opening. It is generally cone-shaped, although it can also be planar. The Luer collar and the Luer cone are formed on the frustoconical portion of the syringe body.

The Luer collar is composed of a tubular portion with an inner thread on its inner face and reinforcing ribs on the outer face. The outer and/or inner face of the Luer collar can have an angle to the longitudinal axis of the syringe of 0°-2°, such that the wall thickness decreases slightly toward the free end. It is thus possible to achieve good demoldablity of the syringe body during production by injection molding, i.e. the syringe body can be easily released from the mold after the injection molding tool has been opened.

The inner thread of the Luer collar can be configured according to standards ISO 594/2 and ISO 80369-7. For example, it can be a staggered right-handed thread. The thread pitch can be 5 mm, in accordance with ISO 594/2, or between 2 mm and 5 mm.

The Luer cone may be configured in accordance with ISO 594/1.

The syringe body and Luer lock connector are formed in one piece. Here, “in one piece” is to be understood as meaning that the syringe body and Luer lock connector are materially connected and made substantially of the same basic material. This can be achieved, for example, by producing the syringe by injection molding. However, it is likewise conceivable for the Luer lock connector to be welded or formed onto a syringe body.

In some embodiments, the Luer collar has between 4 and 24, such as between 8 and 16, reinforcing ribs. The number of the reinforcing ribs is generally such that a uniform strength of the Luer collar is obtained in all directions, although an individual reinforcing rib may have sufficient strength. The Luer collar may, for example, have twelve reinforcing ribs of the same dimensions, which are arranged at uniform angular intervals about the Luer collar. In some embodiments, the reinforcing ribs are all of the same size and are arranged at constant angular intervals about the Luer collar. However, it is equally possible to provide several types of reinforcing ribs having different designs.

In some embodiments, the reinforcing ribs have a radial extent of between 0.5 mm and 2.5 mm at their base and have a lateral extent of between 0.5 mm and 2.0 mm. The base of a reinforcing rib is here to be understood as that region of a reinforcing rib directed toward the frustoconical portion. The reinforcing ribs can taper toward the free end of the Luer collar.

In some embodiments, the reinforcing ribs, starting from the frustoconical portion, extend axially at least as far as half the height of the Luer collar and as far as the upper edge 13 of the Luer collar (illustrated in FIG. 4). The reinforcing ribs thus may have a length corresponding to 50% to 100% of the length of the Luer collar.

In some embodiments, the transition of the reinforcing ribs to the Luer collar and/or to the frustoconical portion is rounded, with a radius of at least 0.3 mm, such as 0.5 to 1.5 mm. By virtue of the rounded transitions, peak loads can be avoided and forces that occur can be distributed more evenly.

In some embodiments, an annular elevation is arranged on the bottom surface between Luer collar and Luer cone and on the outer face of the Luer cone and is designed such that it acts as an abutment for a screw-in attachment. Measured from the lowest point of the bottom surface, the annular elevation can have a height of 0.3 mm to 2 mm and a radial extent of 0.3 mm to 1.5 mm. When an attachment bearing on the Luer cone is screwed in, it will strike against the annular elevation. The annular elevation may have a planar surface.

In some embodiments, the syringe has an annular recess which lies radially in the continuation of the frustoconical portion and axially below the wall of the syringe cone, such that a cylindrical cavity is formed between through-channel and frustoconical portion, the diameter of which cylindrical cavity corresponds approximately to the diameter of the Luer cone and/or the axial extent of which corresponds approximately to the wall thickness of the frustoconical portion. It has been discovered that the fracture resistance of the syringe can be increased by the annular recess, presumably since the axial force transmission from the Luer cone to the frustoconical portion of the syringe body can be reduced. Whereas high axial forces on the Luer cone can build up in the case of some attachments used on the market and can lead to fractures in the frustoconical portion, such fractures occur considerably less often by virtue of the cylindrical cavity.

In some embodiments, the syringe has both an annular elevation and an annular recess as previously described, which are arranged in relation to each other such that the Luer cone is connected to the frustoconical portion substantially via the annular elevation. On the one hand, the axial forces from the Luer cone on the frustoconical portion of the syringe body can thus be further reduced and, on the other hand, the syringe, in a region formed of Luer cone and annular elevation, has a relatively constant wall thickness. This has a positive effect on the injection molding behavior, since sites with a locally greater wall thickness can lead to problems such as sink marks and inner stresses, which can reduce the strength of the syringe. Moreover, by avoiding a locally greater wall thickness, it is possible to achieve a short cooling time and thus a short demolding time.

In some embodiments, the wall thickness in the body region formed of the annular elevation and of the adjoining cone is between 50% and 200% of the wall thickness of the Luer cone, such as between 75% and 125%. The wall thickness of the Luer cone, which typically increases slightly toward the syringe body, is the wall thickness in a portion of the Luer cone near the syringe body.

In some embodiments, the transitions of the edges at the transition of the frustoconical portion to the Luer cone, at the Luer cone, at the transition from the Luer cone to the annular elevation and/or at the annular elevation are rounded, with a radius of at least 0.1 mm, such as of 0.15 mm to 1.0 mm. Stress concentrations in the transition regions can thus be reduced, and fractures running into the transition edges can be avoided or reduced.

In some embodiments, the inner thread of the Luer collar extends as far as the bottom surface. In contrast to an inner thread that terminates above the bottom surface, an inner thread that continues as far as the bottom surface can be produced during injection molding with the aid of a screw core, which is unscrewed for demolding after the injection molding procedure. Production by injection molding is thus readily possible.

In some embodiments, the axial protrusion of the Luer cone above the Luer collar is at least 2.2 mm, such as 2.3 mm. The danger of an attachment being screwed in too far can thus be reduced.

In some embodiments, the syringe body moreover has a flange, which may be configured in one piece with the syringe body. The flange can be cylindrically symmetrical or non-cylindrically symmetrical in order to secure various rotatable or also rotationally fixed finger flanges or backstop devices. Backstop devices prevent a stopper and/or a piston rod from being pulled out of the syringe.

In some embodiments, the syringe moreover comprises a stopper and/or a piston rod, which can be configured according to the prior art. For securing the piston rod to the stopper, the stopper usually has a recess that can be provided with an inner thread into which the stopper is screwed.

Moreover, a pharmaceutical product is also provided according to the present invention. It comprises the syringe as described above. The syringe is filled with a medical liquid, which may contain an active substance. A medical liquid is, for example, a liquid that is to be administered intravenously. Examples of a liquid to be administered intravenously are a saline solution, a glucose solution, a nutrient solution for parenteral nutrition, an emulsion or the like. The active substances can be dissolved and/or dispersed in the medical liquid. An example of an emulsion is an emulsion that contains propofol. Propofol is described by the chemical name 2,6-diisopropylphenol (IUPAC). The syringe provided according to the present invention has proven advantageous for emulsions containing propofol. A syringe thus filled has improved stability even during downstream autoclaving.

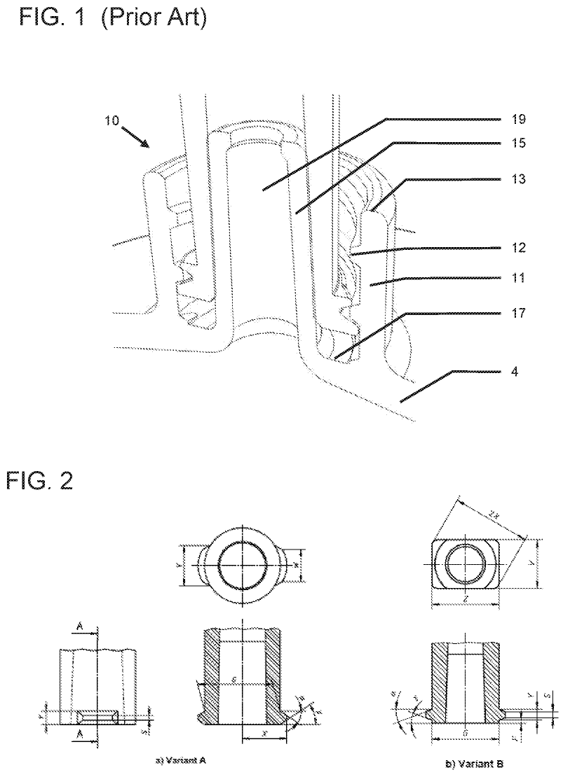

Referring now to the drawings, FIG. 1 shows a perspective sectional view of a Luer lock connector with screwed-in attachment according to the prior art. The Luer cone 15 with the through-channel 19 is located centrally on a frustoconical portion 4 of the syringe body. The Luer collar 11 with an inner thread 12 is arranged around the Luer cone 15. A bottom surface 17, which is planar, is arranged between Luer cone 15 and Luer collar 19. The wall thickness of the syringe body in the region of the bottom surface 17 corresponds approximately to the wall thickness of the syringe body in the frustoconical portion 4.

FIG. 2 shows two different variants A and B of attachments as per ISO 594-2 for joining to a Luer lock connector. For variant A, FIG. 2 shows a longitudinal cross section, a side view, and a view from above. The attachment does not have a continuous outer thread but instead only two opposite portions of an outer thread, which are arranged in a lower portion of the attachment and which can engage in the inner thread 12 of the Luer collar of FIG. 1. For variant B, FIG. 2 shows a longitudinal cross section and a view from above. Again, the attachment does not have a continuous outer thread but instead only four portions of an outer thread, which are arranged in a lower portion of the attachment and which can engage in the inner thread 12 of the Luer collar of FIG. 1.

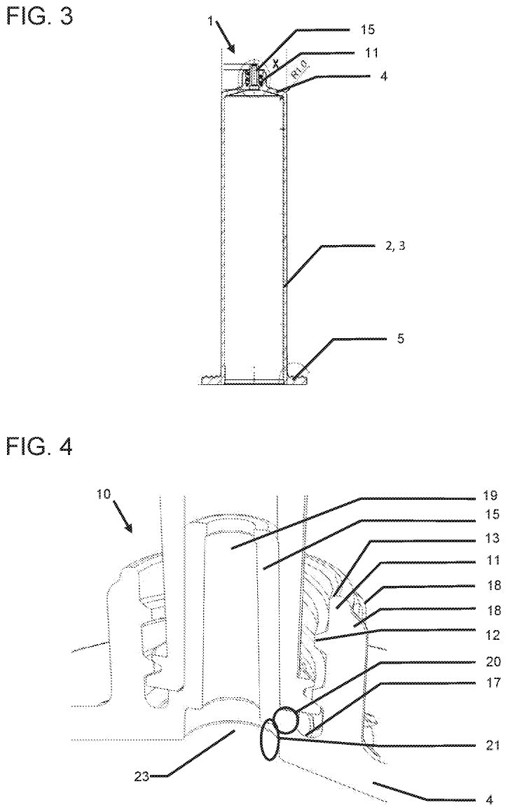

FIG. 3 shows a longitudinal section through an exemplary embodiment of a syringe 1 provided according to the present invention which has a syringe body 2 with a cylindrical portion 3, and with a frustoconical portion 4 in which the syringe body 2 tapers conically toward the through-channel 19. A flange 5 is arranged at the proximal end. The Luer lock connector 10 comprises a Luer cone 15 and a Luer collar 11. The syringe 1 is made of a COC material, and the syringe body 2 has a wall thickness of approximately 1.5 mm in the cylindrical region. The inner face of the syringe body 2 has a lubricant coating with a silicone oil.

FIG. 4 shows a perspective sectional view of the Luer lock connector 10 of an exemplary embodiment of a syringe provided according to the present invention with screwed-in attachment, wherein the cross section runs through reinforcing ribs 18 arranged on the outer face of the Luer collar 11.

The reinforcing ribs 18, starting from the frustoconical portion 4, extend axially along the outer wall of the Luer collar 11 and end underneath the free end of the Luer collar 11. The transition regions between the reinforcing ribs 18 and the frustoconical portion 4 are strongly rounded, with a radius of approximately 1.0 mm, and the transition regions between the reinforcing ribs 18 and the cylindrical portion of the Luer collar 11 are likewise rounded, with a radius of approximately 0.5 mm.

The Luer lock connector 10 moreover comprises the Luer cone 15 with the through-channel 19. A bottom surface 17 is formed between Luer cone 15 and Luer collar 11. The inner thread 12 of the Luer collar 11 extends as far as the bottom surface 17. At the lower end of the Luer cone 15, an annular elevation 20, which is step-shaped in the cross sectional view, is formed on the bottom surface 15. Measured from the lowest point of the bottom surface 17, the annular elevation 20 has an axial height of 1.2 mm, and it has a radial extent of 0.7 mm. Inside the syringe body 2, an annular recess 21 is formed which is arranged approximately in an intersection region of the downwardly continued syringe cone 15 and of the inwardly continued frustoconical portion 4. The Luer cone 15 is connected to the frustoconical portion 4 of the syringe body 2 substantially via the annular elevation 20. The wall thickness in the body region consisting of annular elevation 20 and Luer cone 15 is relatively constant. On account of the annular recess 21, an approximately cylindrical cavity 23 is obtained in the lower region of the through-channel 19 and forms a widening of the through-channel 19 in the lower region thereof. The transition regions on the outer face of the syringe body between Luer cone 15, annular elevation 20, bottom surface 17 and Luer collar are rounded, with radii in the range of 0.15 mm to 0.5 mm. The transition regions on the inner face of the syringe body between Luer cone 15, annular recess 21 and frustoconical portion 4 are likewise rounded, with a radius of approximately 0.2 mm.

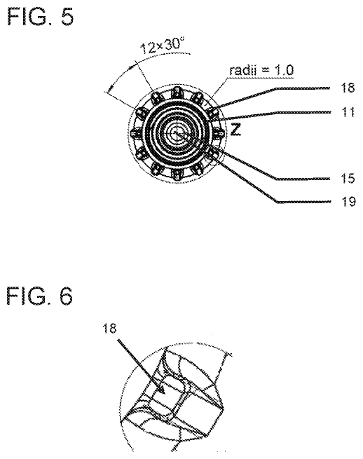

FIG. 5 shows a horizontal cross section through the Luer lock connector 10 of an exemplary embodiment of a syringe provided according to the present invention. On the outer face of the Luer collar 11, twelve reinforcing ribs 18 are arranged at angular intervals of 30° about the Luer collar 11. At their base, the reinforcing ribs 18 have approximately a rectangular cross section with a width of approximately 0.8 mm and a radial dimension of approximately 0.6 mm. The base is understood here as a lower portion of the reinforcing ribs 18 into which the transition radii extend as far as the frustoconical portion 4 but no farther and which, in the longitudinal direction of the syringe, lies approximately at the height of the annular elevation 20.

FIG. 6 shows an enlarged detail from FIG. 5. It can be seen that the reinforcing ribs 18 have, at their foot, rounded transition regions to the frustoconical portion 4, and they also have at their upper end a rounded edge with a radius of approximately 0.2 mm.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

### LIST OF REFERENCE SIGNS

1 syringe
2 syringe body
3 cylindrical portion
4 frustoconical portion
5 finger flange
10 Luer lock connector
11 Luer collar
12 inner thread
13 upper edge of the Luer collar
15 Luer cone
17 bottom surface
18 reinforcing ribs
19 through-channel
20 annular elevation
21 annular recess
23 cylindrical cavity
30 attachment

What is claimed is:

1. A syringe, comprising:
a syringe body including a cylindrical portion and a frustoconical portion adjoined to the cylindrical portion; and
a Luer lock connector including a Luer collar with an inner thread and a Luer cone with a through-channel, the Luer collar and the Luer cone forming a bottom surface therebetween, the syringe body and the Luer lock connector being formed in one piece and comprising a cyclic olefin resin, the Luer collar including reinforcing ribs on its outer face.

2. The syringe of claim 1, wherein the Luer collar has between 4 and 24 reinforcing ribs.

3. The syringe of claim 1, wherein the reinforcing ribs have a radial extent of between 0.5 mm and 2.5 mm at their base and have a lateral extent of between 0.5 mm and 2.0 mm.

4. The syringe of claim 1, wherein the reinforcing ribs, starting from the frustoconical portion, extend axially at least as far as half a height of the Luer collar.

5. The syringe of claim 4, wherein the reinforcing ribs, starting from the frustoconical portion, extend as far as an upper edge of the Luer collar.

6. The syringe of claim 1, wherein a transition of the reinforcing ribs to at least one of the Luer collar or the frustoconical portion is rounded with a radius of at least 0.3 mm.

7. The syringe of claim 1, wherein an annular elevation is arranged on the bottom surface and on the outer face of the Luer cone and is configured to act as an abutment for a screw-in attachment.

8. The syringe of claim 7, wherein an annular recess lies radially in a continuation of the frustoconical portion and axially below a wall of the syringe cone, such that a cylindrical cavity is formed between the through-channel and the frustoconical portion, wherein at least one of:
a diameter of the cylindrical cavity corresponds approximately to a diameter of the Luer cone; or
an axial extent of the cylindrical cavity corresponds approximately to a wall thickness of the frustoconical portion.

9. The syringe of claim 8, wherein the annular elevation and the annular recess are arranged in relation to each other such that the Luer cone is connected to the frustoconical portion substantially via the annular elevation.

10. The syringe of claim 9, wherein a wall thickness in a body region formed of the annular elevation and of the adjoining cone is between 50% and 200% of a wall thickness of the Luer cone.

11. The syringe of claim 9, wherein transitions of edges at least one of at a transition of the frustoconical portion to the Luer cone, at the Luer cone, at a transition from the Luer cone to the annular elevation or at the annular elevation are rounded with a radius of at least 0.1 mm.

12. The syringe of claim 7, wherein the annular elevation includes a planar surface.

13. The syringe of claim 1, wherein the inner thread of the Luer collar extends as far as the bottom surface.

14. The syringe of claim 1, wherein an axial protrusion of the Luer cone above the Luer collar is at least 2.2 mm.

15. The syringe of claim 1, wherein the syringe body further includes a finger flange.

16. The syringe of claim 15, wherein the finger flange is configured in one piece with the syringe body.

17. The syringe of claim 1, further comprising at least one of a stopper or a piston rod.

18. The syringe of claim 1, wherein the syringe is filled with a medical liquid.

19. The syringe of claim 18, wherein the medical liquid comprises propofol.

20. The syringe of claim 1, wherein at least one of the outer face or an inner face of the Luer collar defines an angle to a longitudinal axis of the syringe of 0°-2°.
